(19) United States
(12) Patent Application Publication
(10) Pub. No.: US 2007/0270322 A1
(43) Pub. Date: Nov. 22, 2007

(54) Melding soap bar

(76) Inventor: Chong Wook Pak, Pasadena, CA (US)

Correspondence Address: CHONG WOOK PAK, 8180 MANITOBA STREET, UNIT 211, PLAYA DEL REY, CA, 90293, US

(21) Appl. No.: 11/435,124

(22) Filed: May 16, 2006

(51) Int. Cl. A61K 8/02

(52) U.S. Cl. 510/141

(57) ABSTRACT

A soap bar that can be melded with a used soap bar is disclosed. A meldable soap bar in accordance with the present invention comprises a plurality of projections, cavities, grooves, or any combination thereof.

### BACKGROUND OF THE INVENTION

1. Field of the Invention

This invention relates generally to soap bars, and in particular to a soap bar with projections that facilitates a melding process with a used soap bar.

2. Description of the Related Art

Soaps are generally made with smooth surfaces so as to glide smoothly across the face, body, or hands during washing. As such, there is not much concern with the surface structure of a soap bar except that it is aesthetically pleasing and comfortable when held. Instead focus is directed more towards chemical compositions of the soap so as to provide soaps that cleanse thoroughly, smell pleasantly, and moisturize effectively. However, every soap bar suffers from the same problem. Upon repeated use the soap bar becomes smaller and smaller to a point where it becomes difficult to use. When the soap becomes too small it becomes difficult to lather the body because the hands come into contact with the body more so than the soap bar. This causes increased friction, reduced lathering and frequent dropping, making it difficult to wash. Some people may continue to use the soap until it disintegrates in their hand while others may just throw it away when it becomes more of an inconvenience than an effective soap bar. Still others may try to compress the used soap bar into a new soap bar. However, attempting to squeeze a used, smooth soap bar into a new, smooth soap bar may be problematic in that it requires a lot of pressure. Even if successful, the used soap bar is likely to slip off during use. Thus, what is required is a soap bar that actually melds with a used soap bar when squeezed together without great effort and stays together even when used.

The current invention facilitates the melding of a used soap bar with a meldable soap bar by introducing at least one projection on a surface of a meldable soap bar. By creating at least one projection on the surface of the meldable soap bar, the pliable used soap bar when pressed onto the projections, becomes imbedded in between the spaces created by the projections allowing the projections of the meldable soap bar to effectively “catch” the used soap bar. This prevents the used soap bar from slipping off during further use. In addition, since the used soap bar is melded on the surface of the meldable soap bar, normal use continues the melding process. Thus, through minimal effort an individual can combine a used soap bar and a meldable soap bar into one, thereby effectively and efficiently utilizing the used soap bar to its completion.

### SUMMARY OF THE INVENTION

To minimize the limitations in the prior art, and to minimize other limitations that will become apparent upon reading and understanding the present specification, the present invention discloses a meldable soap bar that facilitates melding a used soap bar into the meldable soap bar. A soap bar in accordance with the present invention comprises at least one projection from a surface. Another embodiment of the present invention comprises at least one projection on a concave surface so as to protect the projection or projections. Another embodiment of the present invention comprises at least one cavity on a surface of the meldable soap bar. A further embodiment of this invention comprises at least one cavity on a surface of the meldable soap bar wherein at least one cavity further comprises at least on projection.

### BRIEF DESCRIPTION OF THE DRAWINGS

Referring now to the drawings in which like reference numbers represent corresponding parts throughout:

FIG. 1 illustrates an isometric view of a meldable soap bar comprising at least one cavity;

FIG. 2A illustrates an isometric view of a meldable soap bar comprising a plurality of projections;

FIG. 2B illustrates a front view a meldable soap bar comprising a plurality of projections wherein the plurality of projections do not protrude out of the cavity;

FIG. 3 illustrates an isometric view of a meldable soap bar comprising a plurality of projections wherein the projections are letters;

FIG. 4 illustrates an isometric view of a meldable soap bar comprising a plurality of cavities wherein the cavities are grooves;

FIG. 5A illustrates a front view of a meldable soap bar comprising a plurality of grooves wherein the plurality of grooves form a plurality of ridges;

FIG. 5B illustrates an isometric view of a meldable soap bar comprising a plurality of grooves wherein the plurality of grooves form a plurality of ridges;

FIG. 6 illustrates an isometric view of a meldable soap bar comprising one set of parallel grooves perpendicular to a second set of parallel grooves;

FIG. 7 illustrates a front view of a meldable soap bar comprising a plurality of projections on a concave face;

FIG. 8 illustrates an isometric view of a meldable soap bar comprising a plurality of projections not inside a cavity;

FIG. 9A illustrates an isometric view of a meldable soap bar comprising a plurality of projections on a concave surface.

FIG. 9B illustrates a front view of a meldable soap bar comprising a plurality of projections on a concave surface.

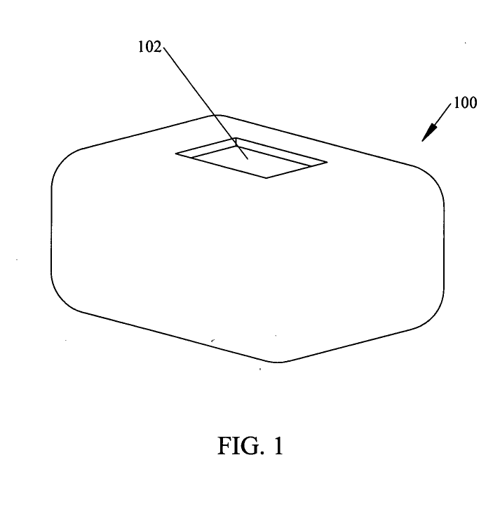

### DETAILED DESCRIPTION OF THE DRAWINGS

In the following description of the preferred embodiment, reference is made to the accompanying drawings that form a part hereof, and in which is shown by way of illustration a specific embodiment in which the invention may be practiced. It is to be understood that other embodiments may be utilized and structural changes may be made without departing from the scope of the present invention.

### Specifics of the Invention

A typical soap bar comprises a substantially smooth surface. An embodiment of the meldable soap bar is a soap bar 100 comprising at least one cavity 102 wherein at least one cavity 102 allows the melding of a used soap bar with the soap bar 100 comprising at least one cavity 102. Melding means the merging of two objects into one. By creating a cavity 102 sufficiently large and sufficiently deep, the used soap bar, when pressed against the soap bar 100 on the surface comprising the cavity 102, will be embedded into the cavity 102 while at least one edge of the cavity 102 will catch the used soap bar. This will cause sufficient friction and prevent the used soap bar from slipping off the meldable soap bar 100. With repeated use, the used soap bar will continue to meld into the meldable soap bar 100. The smaller the used soap bar is the better the melding process will be. Furthermore, optimal results are accomplished if the used soap bar is wet and the meldable soap 100 bar is dry.

Another preferred embodiment of the meldable soap bar is a soap bar 100 comprising at least one cavity 102 wherein at least one cavity 102 further comprises at least one projection 200, 202, 204, 206, 208, 210, 212 that facilitates the melding process. Adding at least one projection 200, 202, 204, 206, 208, 210, 212 within at least one cavity 102 facilitates the melding process by creating more places where the used soap bar can be caught by the meldable soap bar 100, specifically each edge of the cavity 102 and each additional projection 200, 202, 204, 206, 208, 210, 212. This reduces the likelihood that the used soap bar will slip off the meldable soap bar 100 during use. It is preferable that at least one projection within at least one cavity 102 forms a sharp point 200, 204, 210, 212. This further reduces the likelihood that the used soap bar will slip off the meldable soap bar 100 during use. However, at least one projection within at least one cavity 102 can be dull, round or flat 202, 206, 208 without departing from the scope of this invention. In addition, at least one projection 200, 202, 204, 206, 208, 210, 212 within at least one cavity 102 can be in the form of any geometric shape. For example, at least one projection 200, 202, 204, 206, 208, 210, 212 in at least one cavity 102 can be a cone 200, cylinder 202, pyramid 204, cube 206, block 208, ridge 210 or any other geometric shape or any combination of geometric shapes thereof.

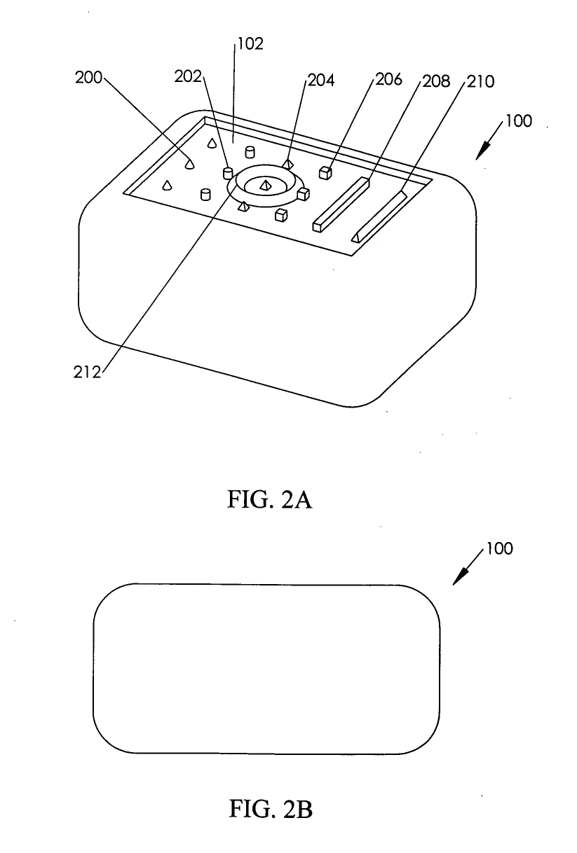

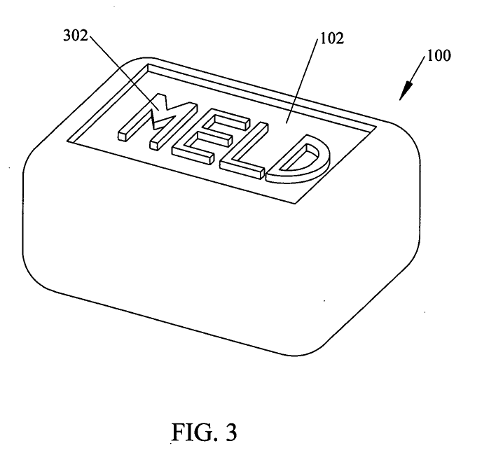

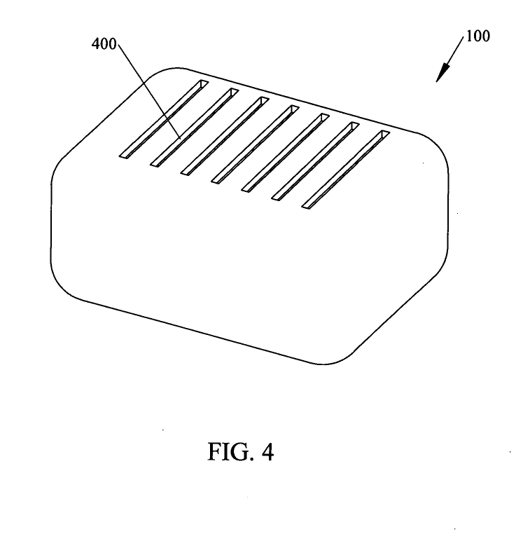

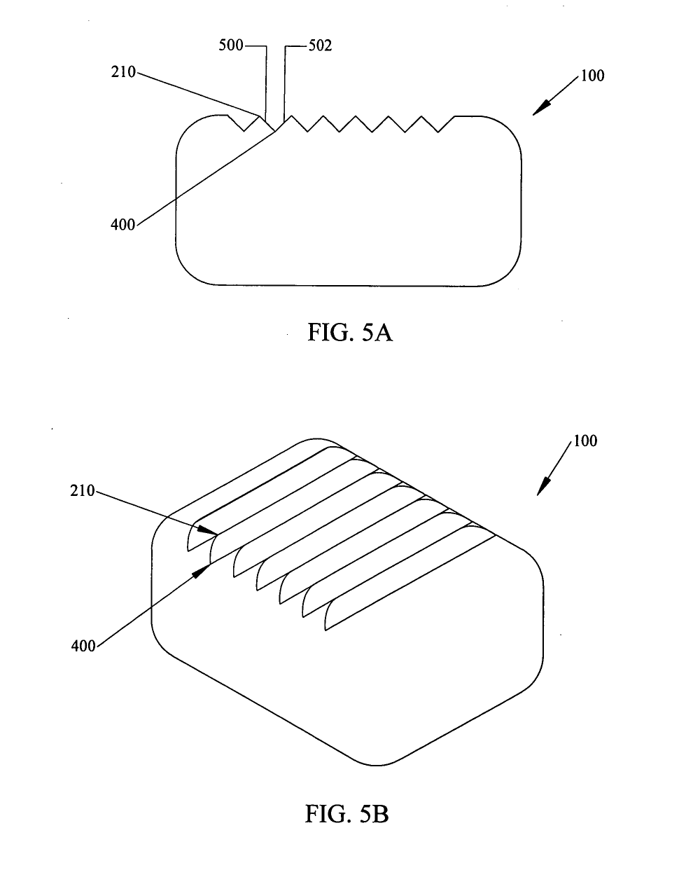

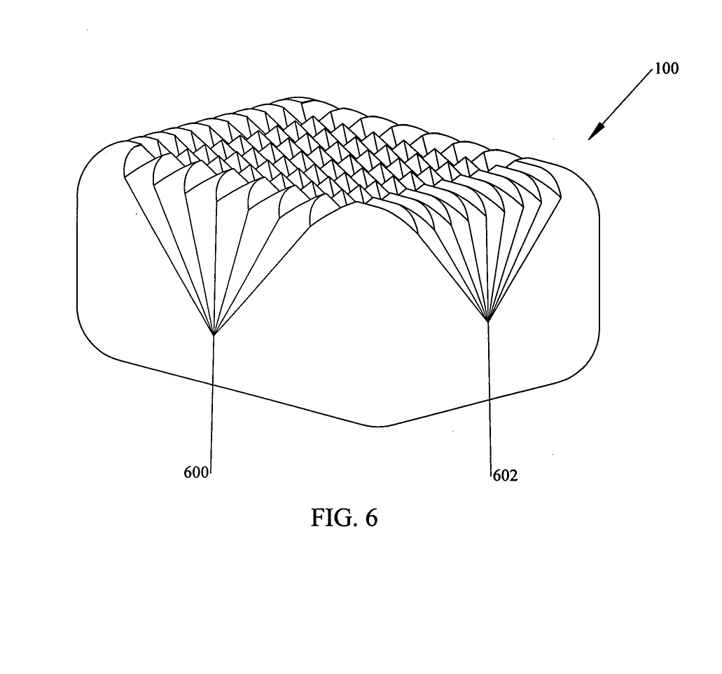

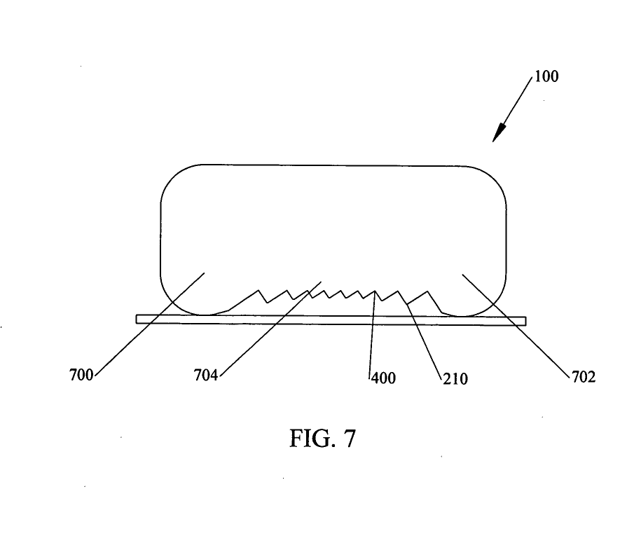

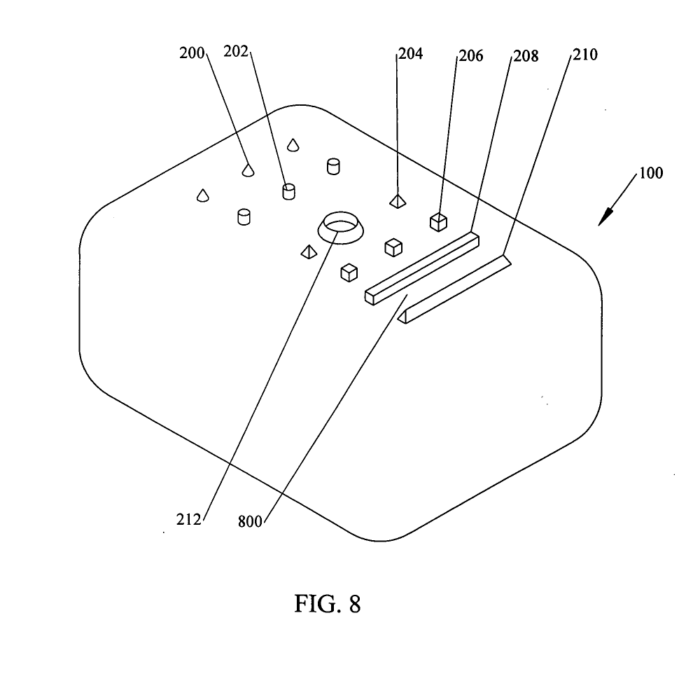

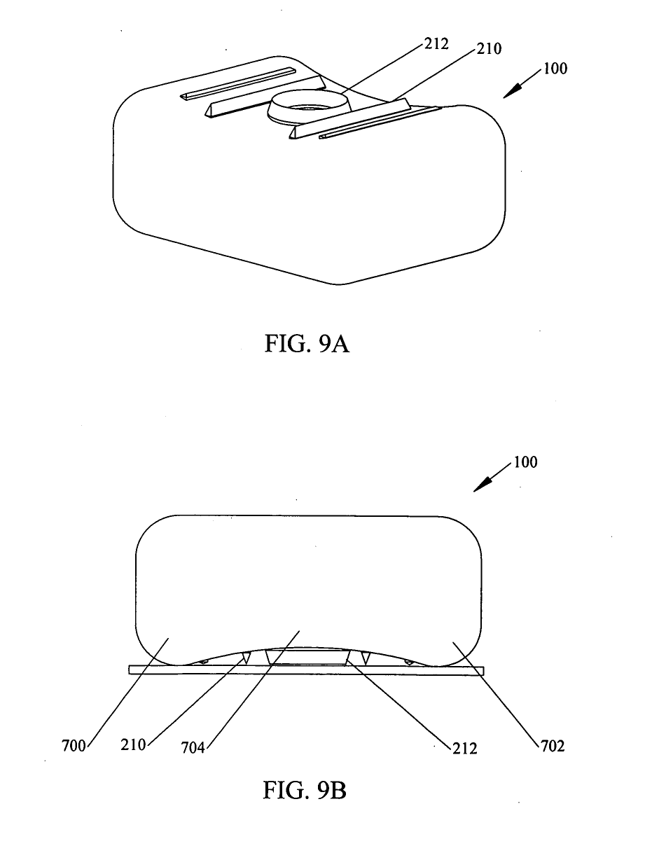

It is preferable that at least one projection 200, 202, 204, 206, 208, 210, 212 does not protrude out of the cavity 102 as shown in the front view of a meldable soap bar in FIG. 2B. Having at least one projection 200, 202, 204, 206, 208, 210, 212 arising from within the cavity 102 but not out of the cavity 102 protects the projections 200, 202, 204, 206, 208, 210, 212 from damage during transportation or storage. Should the side of the meldable soap bar 100 with the cavity 102 come into contact with any flat surface, the meldable soap bar 100 would rest on the outer edges and anything within the cavity 102 would remain protected.

It is preferable that the meldable soap bar comprise a plurality of projections 200, 202, 204, 206, 208, 210, 212 within at least one cavity 102. The plurality of projections 200, 202, 204, 206, 208, 210, 212 within at least one cavity can be arranged to form parallel lines, circles, concentric circles, zig-zags, sine waves, arcs, curves, any other shape or any combination thereof. Furthermore, the plurality of projections 200, 202, 204, 206, 208, 210, 212 can also be arranged to form letters 302, words, numbers, symbols, designs, logos, trademarks, trade names, pictures, communicative messages, artistry, any other shape or any combination thereof. It is most preferable that the projections 200, 202, 204, 206, 208, 210, 212 do not protrude out of at least one cavity 102.

It is preferable that the length and width of the cavity 102 is inversely proportional to the number of cavities 102 present. In other words, the fewer number of cavities 102 present, the larger the cavities 102 can be. The higher the number of cavities 102 present, the smaller the size of the cavities 102 can be. A preferred embodiment of the meldable soap bar is a soap bar 100 comprising at least one cavity 102 wherein at least one cavity 102 is at least one millimeter in depth with a length and width slightly smaller than the size of the meldable soap bar 100. At least one cavity 102, at least one millimeter in depth, can also be any geometric shape, symbol, letter, word, number, design, logo, trademark, trade name, picture, communicative message, artistry, any other shape or any combination thereof.

It is more preferable that the meldable soap bar 100 comprises a plurality of cavities 102. The plurality of cavities 102 can be arranged to form a single line, non-parallel lines, parallel lines, circles, concentric circles, zig-zags, sine waves, arcs, curves, or any other shape and design or any combination thereof. Another embodiment of this invention is to make the plurality of cavities narrow so as to form a plurality of grooves 400. The plurality of grooves can be created with at least two walls and preferably four walls. It is preferable that at least two opposite walls 500, 502 of a groove 400 are non-parallel to each other, specifically, where at least a first wall 500 slants away from a second wall 502. Having at least two grooves with slanting walls adjacent to each other results in the creation of a ridge 210 between the two grooves 400. It is preferable to have a plurality of grooves 400 with slanting walls 500, 502 adjacent to each other forming a plurality of ridges 210. The apex of the plurality of ridges 210 can be pointed, flat, or rounded. Another embodiment of the meldable soap bar is a soap bar 100 comprising a first set of a plurality of parallel grooves 600 and a second set of a plurality of parallel grooves 602 wherein the second set of a plurality of parallel grooves 602 is perpendicular to the first set of a plurality of parallel grooves 600. Such an arrangement can create a plurality of pyramids, blocks, trapezoids, and other polygonal shapes. The plurality of grooves can also be arranged to form letters, words, numbers, symbols, designs, logos, trademarks, trade names, pictures, communicative messages, artistry, any other shape or any combination thereof.

It would be preferable for the apex of the ridges 210 to be below at least two opposite and outer edges 700, 702 of the meldable soap bar 100. This would provide protection for the ridges 210 during transportation or storage. This can be created by making a soap bar 100 comprising a concave surface 704 and a plurality of grooves 400 wherein the plurality of grooves 400 is on the concave surface 704.

Another embodiment of a meldable soap bar is a soap bar 100 comprising at least one projection 200, 202, 204, 206, 208, 210, 212, wherein at least one projection 200, 202, 204, 206, 208, 210, 212 allows the melding of a used soap bar with the meldable soap bar 100 comprising at least one projection 200, 202, 204, 206, 208, 210, 212. Therefore, melding a used soap bar with a meldable soap bar 100 essentially merges the two bars of soap into one soap bar. At least one projection 200, 202, 204, 206, 208, 210, 212 can be in the form of any geometric shape. For example, at least one projection 200, 202, 204, 206, 208, 210, 212 can be a cone 200, cylinder 202, pyramid 204, cube 206, block 208, ridge 210 or any other geometric shape or any combination of geometric shapes thereof. Preferably, at least one projection forms a sharp point 200, 204, 210, 212. A sharp point would best facilitate the melding of a used soap bar with a meldable soap bar 100 because the sharp point or points could embed more easily into the used soap bar. However, at least one projection can be dull, rounded, or flat 202, 206, 208 without departing from the scope of this invention. This is because the mere presence of a projection provides a point of contact for the used soap bar to catch. A sharp point can be created by making at least one wall of the projection sloped at an angle, like a right triangle, or both walls sloping away from each other like an isosceles or equilateral triangle.

Although this invention can be created with one projection 200, 202, 204, 206, 208, 210, 212 a preferred embodiment of a meldable soap bar is a soap bar 100 comprising a plurality of projections 200, 202, 204, 206, 208, 210, 212. Increasing the number of projections 200, 202, 204, 206, 208, 210, 212 increases the points of contact thereby increasing the ability to catch the used soap bar. Furthermore, increasing the number of projections also inherently generates empty spaces 800, which would serve as a functional equivalent to cavities, into which the used soap bar can be embedded or wedged. Embedding the sharp points into the used soap bar and embedding the used soap bar into the empty spaces 800 would prevent the used soap bar from slipping off the meldable soap bar 100 with the plurality of projections 200, 202, 204, 206, 208, 210, 212.

It is preferable that the plurality of projections 200, 202, 204, 206, 208, 210, 212 traverse substantially across a length and width of the meldable soap bar 100. It is preferable that the plurality of projections 200, 202, 204, 206, 208, 210, 212 cover substantially an entire surface of a soap bar. This will increase the resistance between the used soap bar and the meldable soap bar 100 with a plurality of projections 200, 202, 204, 206, 208, 210, 212 by maximizing the surface area of the used soap bar that comes in contact with the plurality of projections 200, 202, 204, 206, 208, 210, 212, thereby maximizing the amount of used soap that gets embedded in the soap bar with the plurality of projections 200, 202, 204, 206, 208, 210, 212. However, the plurality of projections 200, 202, 204, 206, 208, 210, 212 do not have to cover substantially the entire surface of the meldable soap bar 100. The plurality of projections 200, 202, 204, 206, 208, 210, 212 can be localized in a center of a meldable soap bar 100, at at least one of the ends of the meldable soap bar 100 or any combination of locations on the meldable soap bar 100.

The plurality of projections 200, 202, 204, 206, 208, 210, 212 can be arranged to form a plurality of geometric shapes. For example, the plurality of projections 200, 202, 204, 206, 208, 210, 212 can be arranged to form parallel lines, circles, concentric circles, polygons, zig-zags, sine waves, arcs, curves, any other shape or any combination thereof. Furthermore, the plurality of projections 200, 202, 204, 206, 208, 210, 212 can also be arranged to form letters, words, numbers, symbols, designs, logos, trademarks, trade names, pictures, communicative messages, artistry, any other shape or any combination thereof.

The plurality of projections can also be lowered below at least two opposite edges 700, 702 of a meldable soap bar 100 so as to protect the projections during transportation or storage. This can be accomplished by creating a soap bar 100 comprising a concave surface 704 wherein the concave surface 704 further comprises a plurality of projections 200, 202, 204, 206, 208, 210, 212 and the plurality of projections 200, 202, 204, 206, 208, 210, 212 do not extend above at least two opposite edges 700, 702 of the soap bar 100. During transportation or storage, the surface of the meldable soap bar 100 comprising the plurality of projections 200, 202, 204, 206, 208, 210, 212 may be face down on a flat surface. If the meldable soap bar has a concave surface then the meldable soap bar 100 rests on its edges 700, 702 and if the plurality of projections 200, 202, 204, 206, 208, 210, 212 do not extend passed at least two opposite edges 700, 702 then the plurality of projections 200, 202, 204, 206, 208, 210, 212 will remain undamaged.

The meldable soap bar 100 can be made by first making a soap bar then carving out the type of projection 200, 202, 204, 206, 208, 210, 212 or grooves 400 or cavities 102 desired. Alternatively, the meldable soap bar can be created using an appropriate mold for the desired projections, grooves, or cavities.

It should be noted that the meldable soap bar 100 can be used independently without melding it with a used soap bar. At first a concern may arise that such usage would cause discomfort with the presence of the projection, however, rubbing the projections with the hands in water during normal washing will quickly dull the sharp projections and not cause any discomfort.

### CONCLUSION

In summary, the present invention provides a soap bar that can easily be melded with a used soap bar. A soap bar in accordance with the present invention comprises at least one projection and preferably a plurality of projections, at least one groove and preferably a plurality of grooves, at least one cavity, or any combination thereof.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

What is claimed is:

1. A soap bar comprising at least one cavity, wherein at least one cavity allows the melding of a used soap bar with the soap bar comprising at least one cavity.

2. The soap bar of claim 1, wherein at least one cavity further comprises at least one projection that facilitates the melding process.

3. The soap bar of claim 2, wherein at least one projection does not protrude out of the cavity.

4. The soap bar of claim 3, wherein at least one projection forms a sharp point.

5. The soap bar of claim 4, wherein at least one cavity is at least one millimeter in depth.

6. The soap bar of claim 1, comprising a plurality of cavities.

7. The soap bar of claim 6, wherein the plurality of cavities are a plurality of grooves.

8. The soap bar of claim 7, wherein the plurality of grooves form a plurality of ridges.

9. The soap bar of claim 8, wherein the plurality of ridges form a sharp point.

10. The soap bar of claim 9, wherein the plurality of ridges are on a concave surface.

11. A soap bar comprising at least one projection, wherein at least one projection allows the melding of a used soap bar with the soap bar comprising at least one projection.

12. The soap bar of claim 11 comprising a plurality of projections.

13. The soap bar of claim 12, wherein the plurality of projections form a sharp point.

14. The soap bar of claim 13, wherein the plurality of projections are a plurality of ridges.

15. The soap bar of claim 14, wherein the plurality of ridges forms at least one circle.

16. A soap bar comprising a concave surface, wherein the concave surface further comprises at least one projection wherein at least one projection allows the melding of a used soap bar with the soap bar comprising a concave surface.

17. The soap bar of claim 16 comprising a plurality of projections.

18. The soap bar of claim 17, wherein the plurality of projections form a sharp point.

19. The soap bar of claim 18, wherein the plurality of projections are a plurality of ridges.

20. The soap bar of claim 19, wherein the plurality of ridges forms at least one circle.
